(19) United States
(12) Patent Application Publication
(10) Pub. No.: US 2006/0030746 A1
(43) Pub. Date: Feb. 9, 2006

(54) Linear blood pump

(76) Inventor: Marius Grossmann, Goettingen (DE)

Correspondence Address: SMITH, GAMBRELL & RUSSELL, LLP, 1850 M STREET, N.W., SUITE 800, WASHINGTON, DC, 20036, US

(21) Appl. No.: 10/913,427

(22) Filed: Aug. 9, 2004

(51) Int. Cl. A61N 1/362

(52) U.S. Cl. 600/16

(57) ABSTRACT

A linear blood pump (100) includes a tube (1) having an inner wall, an inner diameter, an axial length and a constant portion. The inner wall is designed and arranged such that blood contacting the inner wall may flow though the tube (1). The constant portion is designed to extend along at least a part of the axial length of the tube (1) and to have a constant inner diameter. At least one check valve (3) is arranged in the constant portion. The check valve (3) is designed and arranged to reciprocate in the constant portion. The blood pump (100) has an especially advantageous flow profile. The blood pump (100) may be used in many fields and for many purposes, for example as a permanent heart supporting system or for blood circulation outside of the human body. The blood pump (100) is especially helpful to guarantee as few blood damage and low emboli rates as possible.

### FIELD OF THE INVENTION

The present invention generally relates to a blood pump. Such blood pumps are especially used to treat hard weakness of human patients and to realize a blood circuit inside and outside of the human body, for example in the sense of a heart-lung machine or dialysis or ventricular assist devices.

### BACKGROUND OF THE INVENTION

Different types of blood pumps have been used in the past to treat hard weakness of human patients and to realize a blood circuit inside of the human body, for example in the sense of a heart-lung machine or dialysis. Especially, pumps making use of rotating and membrane pump principal have been preferred. A substantial drawback of such known blood pumps is the danger of mechanical damage of the cellular and plasmatic components of the blood. There also is the danger of blood clots which enter the human body and which may cause damage.

A blood pump is known from U.S. Pat. No. 5,147,281. The pump includes a movable first tube section in which a first check valve is fixedly arranged. The pump further includes a stationary second tube section in which a second check valve is fixedly arranged. A flexible connecting hose is arranged between the two tube sections, and it interconnects the two tube sections. The connecting hose is designed as a flexible bellow which allows for movement of the movable first tube section including the first check valve with respect to the stationary second tube section and thus the second check valve. This relative movement between the two check valves results in the desired pumping effect. During this movement, the axial length and the shape of the flexible bellow change. Depending on the length of the flexible bellow, the continuous channels located at the inner surface of the bellow increase and decrease, respectively. These channels result in an uneven shape of the inner surface of the flexible bellow. This uneven shape may cause turbulent flow of the blood through the pump and blood accumulating in these channels. As a result, blood cells may be destroyed, and there is the danger all of undesired blood clot.

Another blood pump including a flexible bellow is known from U.S. Pat. No. 4,334,180.

An apparatus for conveying blood is known from German patent No. DE 34 28 828 C2 corresponding to European patent No. EP 0 191 071 B1. The apparatus includes a pump and a separate pump chamber. The pump chamber includes two elastic membranes which divide the pump chamber into an outer part and an inner part. The outer part of the pump chamber is filled with a working fluid. The inner part of the pump chamber is filled with blood. When the working fluid is pumped into the outer part of the pump chamber, blood is displaced from the inner part of the pump chamber. The pump for pumping the working fluid into the outer part of the pump chamber may be designed as a piston pump.

It is known from “Mechanical effects on rates of hemolysis”; Toro Nakahara and Fumitake Yoshida; Journal of Biomedical Materials Research, Vol. 20, 363-374 (1986) that an important mechanical factor controlling rates of hemolysis in practical devices, in which blood does not flow in thin channels or capillaries, is the turbulent stress in the bulk blood, rather than the shear at the blood-solid interface.

### SUMMARY OF THE INVENTION

The present invention relates to a linear blood pump. The blood pump includes a tube having an inner wall, an inner diameter, an axial length and a constant portion. The inner wall is designed and arranged such that blood contacting the inner wall may flow though the tube, the inner wall being made of a material selected from the group consisting of pyrolytic graphite and titanium alloys. The constant portion is designed to extend along at least a part of the axial length of the tube and to have a constant inner diameter. At least one check valve is arranged in the constant portion. The check valve is designed and arranged to reciprocate in the constant portion.

As it is known in the art, the pyrolytic graphite may be produced using a high temperature chemical vapor Deposition (CVD) process. Instead, the tube and its inner wall, respectively, may also be made of a composite material. The term composite material is to be understood herein to at least include composite fiber materials and carbon fiber reinforced plastics.

The present invention also relates to a linear blood pump system. The linear blood pump system includes a blood pump and at least one sensor. The blood pump includes a tube having an inner wall, an inner diameter, an axial length and a constant portion. The inner wall is designed and arranged such that blood contacting the inner wall may flow though the tube. The constant portion is designed to extend along at least a part of the axial length of the tube and to have a constant inner diameter. At least one check valve is arranged in the constant portion. The check valve is designed and arranged to reciprocate in the constant portion. The at least one sensor is designed and arranged to sense at least one value selected from the group consisting of position, velocity and acceleration of the check valve.

The novel blood pump has an especially advantageous flow profile. The blood pump may be used in many fields and for many purposes, for example as a permanent heart supporting system or for blood circulation outside of the human body. The blood pump is especially helpful to guarantee as few blood damage and low emboli rates as possible.

Preferably, at least the inner surface of the tube and the movable valve parts of the check valves are made of a biologically compatible material, especially pyrolytic graphite or titanium nitride. These materials have been found to be chemically inert such that as few as possible deposit of components of the blood at the surfaces of the tube and of the valves takes place. In addition, these materials are lightweight, solid, hard, and they may be processed with low tolerances. It is also possible that the entire tube is made of these materials. Its outer surface can also be coated.

During a pump cycle, a blood pump has to overcome varying viscose and plastic resistance. Consequently, with the novel pump system, it is desired to adapt the movement of the movable valve and the pumping action resulting therefrom to these varying conditions. The power consumption of the drive of the pump system does not correspond to the pump volume, but rather to the pump pressure. Since especially the pump volume, the volume dynamic and the pressure dynamic are important features when supporting the heart of a patient with a blood pump, it is desired to securely control these features.

Other features and advantages of the present invention will become apparent to one with skill in the art upon examination of the following drawings and the detailed description. It is intended that all such additional features and advantages be included herein within the scope of the present invention, as defined by the claims.

### BRIEF DESCRIPTION OF THE DRAWINGS

The invention can be better understood with reference to the following drawings. The components in the drawings are not necessarily to scale, emphasis instead being placed upon clearly illustrating the principles of the present invention. In the drawings, like reference numerals designate corresponding parts throughout the several views.

FIG. 1 is a side view of a first exemplary embodiment of the novel blood pump.

FIG. 2 is a cross-sectional view of the blood pump according to FIG. 1.

FIG. 3 is a side view of a second exemplary embodiment of the novel blood pump.

FIG. 4 is a side view of a third exemplary embodiment of the novel blood pump.

FIG. 5 is a schematic diagram of components of the control of the novel blood pump.

FIG. 6 is a side view of a fourth exemplary embodiment of the novel blood pump.

### DETAILED DESCRIPTION

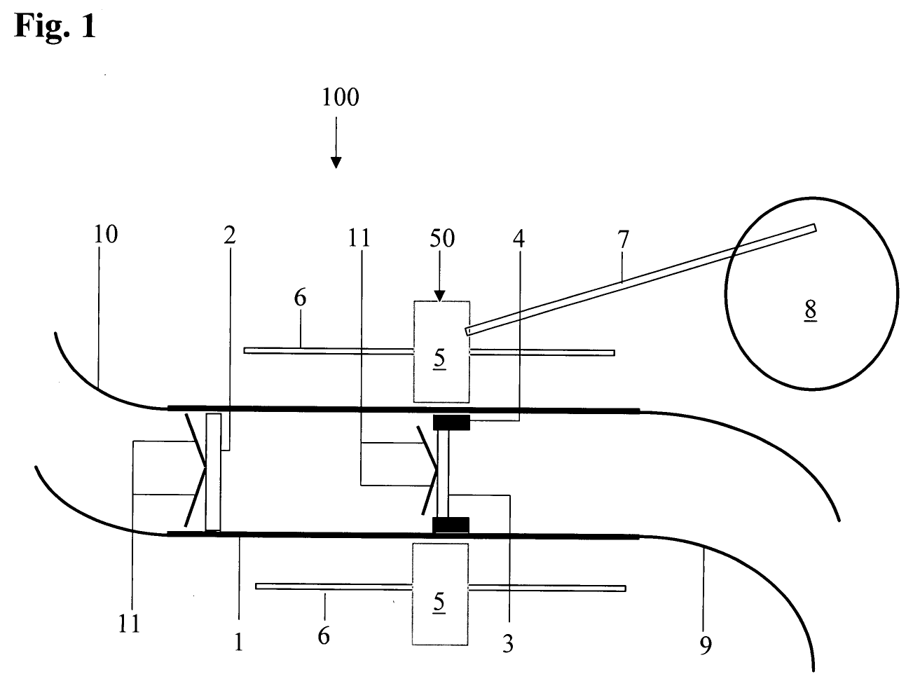

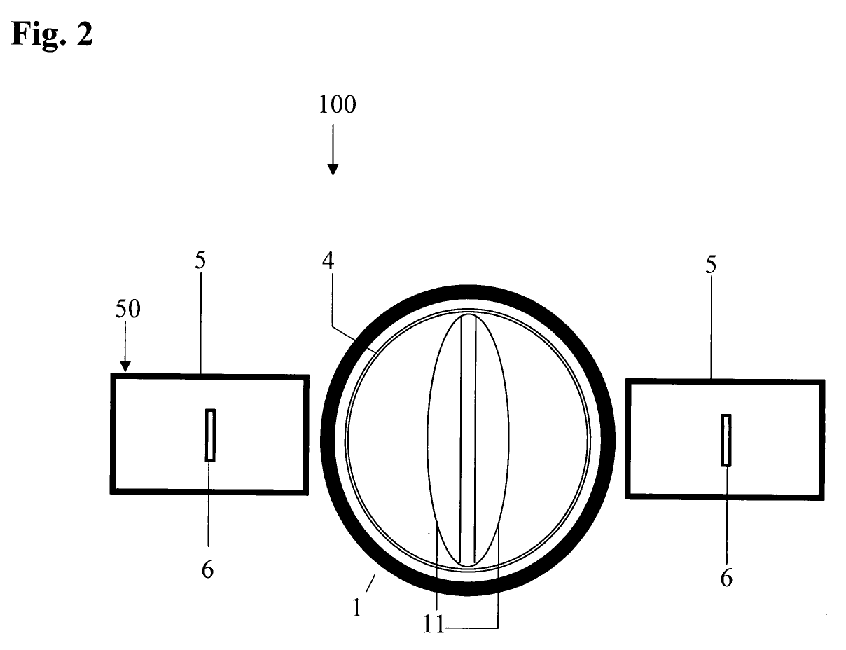

Referring now in greater detail to the drawings, FIGS. 1 and 2 illustrate a blood pump 100 including a tube 1 made of biologically compatible material. Preferably, the tube 1 is either made of pyrolytic graphite or titanium nitride. The tube 1 has an inner diameter of approximately 30 mm and a length of approximately 100 mm. At one end of the tube 1, a mechanical heart valve 2 is fixedly arranged in the tube 1. The heart valve 2 includes two movable valve parts 11 which are connected to a common axle located in the center of the heart valve 2. In this way, the two valve parts 11 may be pivoted to open and close, respectively, the heart valve 2. A second heart valve 3 is also located inside of the tube 1. The second heart valve 3 may has the same design as the heart valve 2. However, it is fixedly connected to a ring 4. Preferably, the ring 4 is made of metal. The outer diameter and the width of the ring 4 are chosen to cooperate with the inner diameter of the tube 1 such that the heart valve 3 may securely slide in the tube 1 without tilting. In this way, the heart valve 3 can move back and forth in the tube 1 in the direction of the longitudinal axis of the tube 1. In other words, the heart valve 3 reciprocates in the tube 1. Both heart valves 2 and 3 are designed as check valves, meaning they exclusively allow for flow of blood through the valves 2, 3 in one direction. Both valves 2, 3 are arranged in the tube 1 in the same sense of direction corresponding to the pumping direction.

The pump 100 further includes a drive 50 serving to move the movable heart valve 3 with respect to the stationary heart valve 2. The drive 50 includes two magnets 5 being arranged outside of the tube 1. The drive 50 further includes two bars 6 serving to guide the magnets 5 during their movement in the longitudinal direction of the tube 1. The two magnets 5 are fixedly interconnected (not illustrated). The drive 50 also includes a rod 7 being connected to an eccentric disk 8 which is rotatably driven by an electric motor (not illustrated). In this way, the rotational movement of the eccentric disk 8 is transferred by the rod 7 and the bars 6 into a linear reciprocating movement of the magnets 5. Due to the magnetic force of the magnets 5, the metal ring 4 and thus the heart valve 3 is moved along with the magnets 5. A first flexible hose 9 is fixedly connected to the first end of the tube 1. For example, the free end of the hose 9 is located in the left pre-chamber of the heart. The blood then enters the tube 1 through the hose 9. A second flexible hose 10 is fixedly connected to the second end of the tube 1. For example, the free end of the hose 10 is located in a big body artery. In this way, the blood does not flow through the left heart chamber such that the left heart chamber relieved. In another embodiment of the pump 100, the drive 50 includes a plurality of electromagnets located in series which are respectively turned on and off to move the movable valve 3 inside of the tube 1. The drive 50 may also be designed to include a coil which moves a magnetic ring being connected to the movable valve 3 in the electromagnetic field of the coil.

At the beginning of a pumping cycle of the pump 100, the movable valve 3 is first moved towards the stationary valve 2. During this first movement, due to the pressure difference upstream and downstream (FIG. 1: right and left) of the respective valve 2, 3, the valve 3 is closed, while the valve 2 is opened. Blood is actively moved in the pumping direction and exits through the hose 10. Consequently, additional blood may flow through the hose 9 into the pump 100. When the moving direction changes at the upper dead center of the path of movement of the valve 3, the pressure also changes and the stationary valve 2 is closed, while the movable valve 3 is opened. When the valve 3 moves away from the valve 2 to reach the lower dead center of the path of movement, the pressure again changes. Thus, the movable valve 3 is closed, while the stationary valve 2 is opened. Another pumping cycle begins.

A special advantage of the novel pump 100 is the almost laminar flow profile. In addition, possible depositions of components of the blood at the inner wall of the tube 1 are removed due to the movement of the ring 4 in the tube 1 at a very early stage such that they do not cause damage in the organism. This advantage may even be increased when using a design of the pump in which both valves are moved. Another special advantage is the possibility of controlling the pump mechanism. By using at least one sensor, it is possible to drive the pump 100 to be volume controlled or pressure controlled. Even the combination of both is possible.

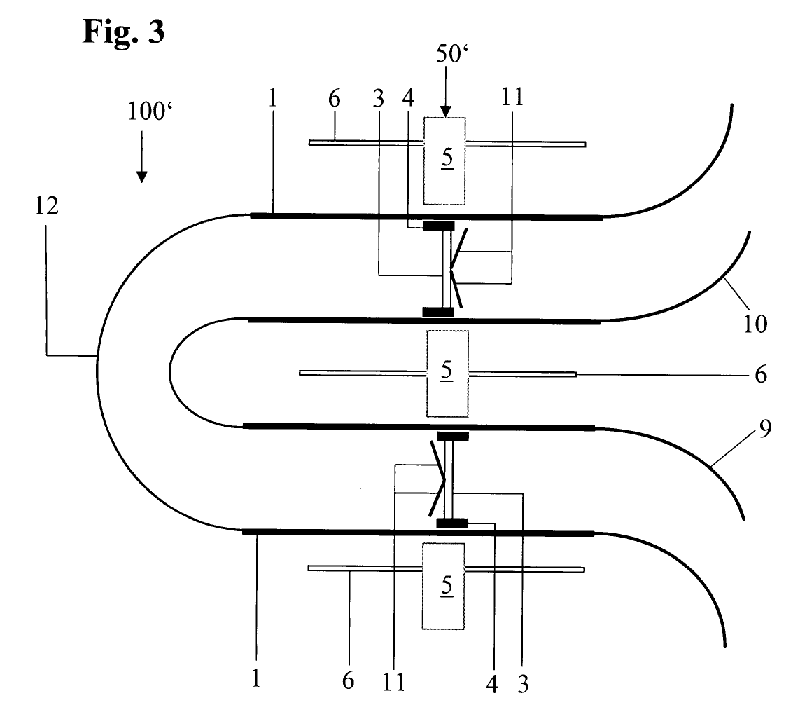

FIG. 3 illustrates a blood pump 100′ including a tube 1 made of biologically compatible material. Preferably, the tube 1 is either made of pyrolytic graphite or titanium nitride. The design and functionality of the blood pump 100′ has a lot in common with the pump 100 described with respect to FIGS. 1 and 2. Thus, it is referred to the above description. In contrast to the pump 100, the pump 100′ does not include one generally straight tube, but rather a tube designed to be U-shaped and to include a first straight tube portion 1, a second straight portion 1 and a bent portion 12. The first and second straight tube portions 1 are interconnected by the bent tube portion 12. The first movable check valve 3 is located in the first straight tube portion 1. A second movable check valve 3 is located in the second straight tube portion 1.

At the beginning of a pumping cycle of the pump 100′, both movable valves 3 are simultaneously moved to the left. Thus, the movable valves 3 approach in the sense of the volume of the tube 1 located between them being decreased due to the U-shape of the tube 1. During this first movement, due to the pressure difference upstream and downstream (FIG. 3: right and left) of the respective valves 3, the first valve 3 (FIG. 3: the lower valve 3) is closed, while the second valve 3 (FIG. 3: the upper valve 3) is opened. Blood is actively moved in the pumping direction and exits through the hose 10. When the moving direction changes at the upper dead center of the path of movement of the valves 3, the pressure also changes and the second valve 3 is closed, while the first valve 3 is opened. Consequently, additional blood may flow through the hose 9 into the pump 100′. When the valves 3 move to the right to reach the lower dead center of the path of movement, the pressure again changes. Thus, the first valve 3 is closed, while the second valve 3 is opened. Another pumping cycle begins. This U-shaped design provides for the special advantage of the length of the blood pump 100′ being decreased such that the blood pump 100′ is especially suitable to be implanted in an organism.

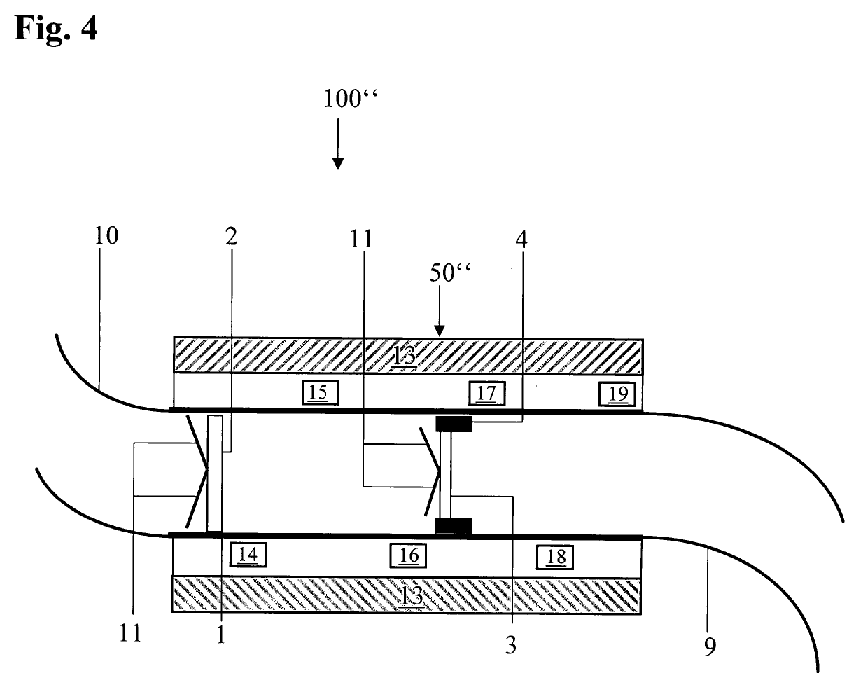

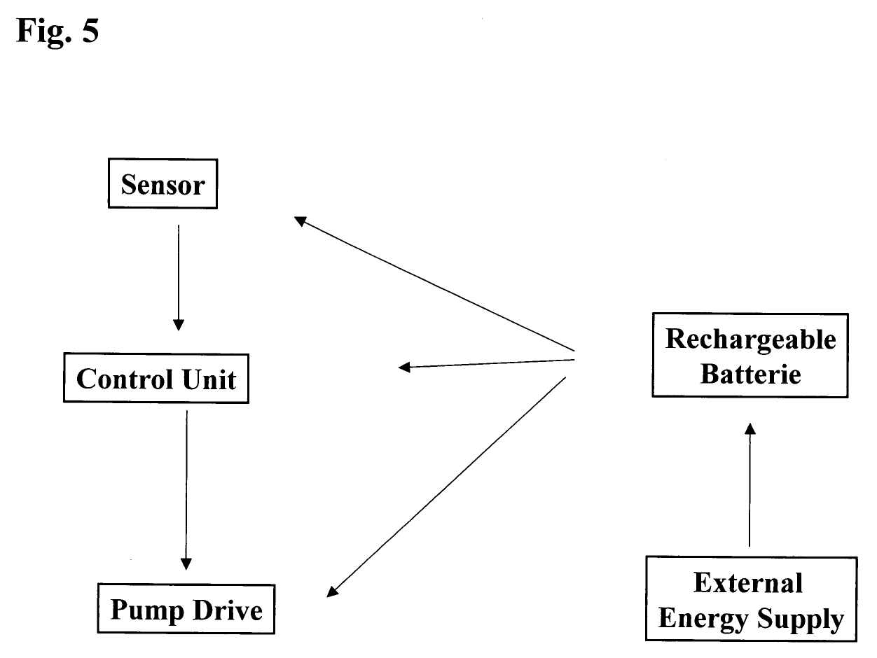

FIG. 4 illustrates another blood pump 100″ of which the design and functionality has a lot in common with the pump 100 described with respect to FIGS. 1 and 2. Thus, it is referred to the above description. In contrast to the pump 100, the pump 100″ includes a different drive 50″. The drive 50″ includes a linear induction motor 13 being designed as a synchronous motor. It could also be designed as an asynchronous motor. The drive 50″ further includes a plurality of sensors 14-19. The sensors 14-19 are designed as Hall sensors, and they are spaced apart along the length of the constant portion of the tube 1 and on both sides of the tube 1. The Hall sensors use the sensing principle of induced voltage. The drive 50″ is designed and arranged to reciprocate the check valve 3 in the constant portion in response to the signals of the sensors 14-19. For this purpose, the ring 4 to which the movable valve 3 is fixedly connected is designed as a permanent magnet such that it can be driven by the linear induction motor 13. The sensors 14-19 serve to sense the position and velocity of the movable valve 3. An electronic control unit (FIG. 5) serves to determine the velocity of the movable valve 3 based on the signals. The electronic control unit controls the linear induction motor 13 based on the determined velocity and position of the movable valve 3. In this way, the stroke and the velocity of the movable valve 3 may be controlled to change (or to keep constant) the amount of the blood which is pumped and the velocity of the blood which is pumped with the pump 100″. Since the design and functionality of linear induction motors and Hall sensors are generally known in the field of electrical engineering, they are not described herein in greater detail.

FIG. 5 schematically illustrates the components of a novel blood pump system. The pump drive drives the movable valve inside of the tube of the pump in response to a control signal generated by the control unit. The control signal is generated based on the signals of the sensors. The system further includes an external energy supply and a rechargeable battery. The external energy supply serves to supply the pump drive and the control unit with current during normal conditions. For example, the external energy supply may be connected to the components to be supplied by an electric line. It is also possible to use electromagnetic induction of an implanted receiver unit instead. The rechargeable battery serves to buffer peaks of power consumption and to allow for temporary disconnection or failures of the external energy supply. Preferably, the rechargeable battery is implanted in the organism.

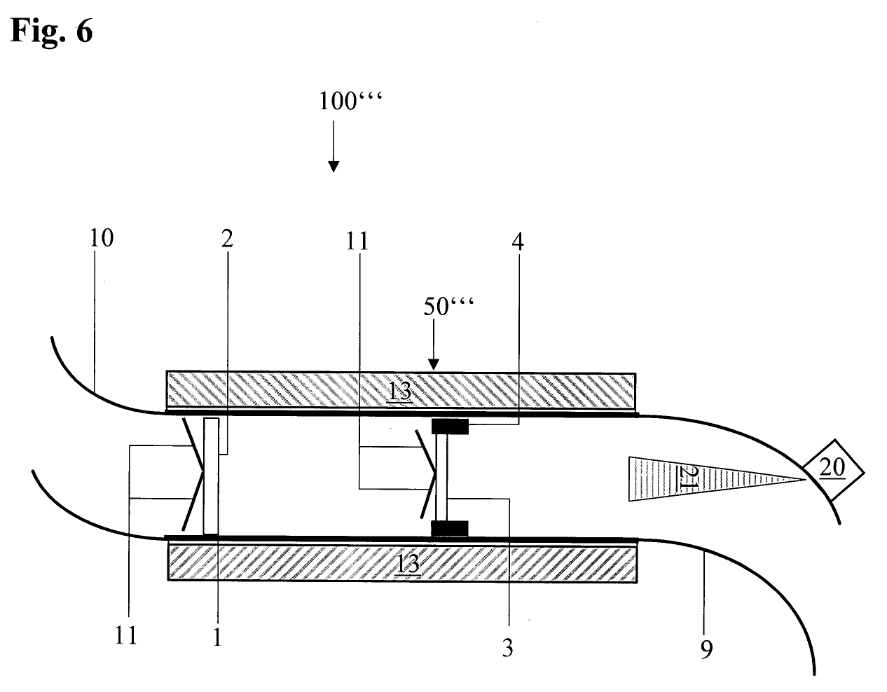

FIG. 6 illustrates another blood pump 100′″ of which the design and functionality has a lot in common with the pump 100″ described with respect to FIG. 5. Thus, it is referred to the above description. In contrast to the pump 100″, the pump 100′″ includes a different drive 50′″. The drive 50′″ also includes the linear induction motor 13, but instead of Hall sensors, it includes an ultrasonic transceiver unit 21. The ultrasonic transceiver unit 21 sends out a pulsed ultrasonic signal onto the movable valve 3 by an ultrasonic window 21. Based on the received ultrasonic signal, the position of the movable valve 3 may be determined. The velocity of the movable valve 3 may be determined by means of the Doppler effect caused by the movement of the movable valve 3. Consequently, the linear induction motor 13 may be controlled as described hereinabove. Since the design and functionality of ultrasonic transceiver unit are generally known in the field of medical engineering, they are not described herein in greater detail.

It is also possible cardially arrange an electrode for use in combination with an electrocardiogram. In this way, it is possible to synchronize the pulse wave of the blood pump with the simultaneously pumping heart of the patient to attain optimal relieve of the heart muscle of the heart of the patient.

Many variations and modifications may be made to the preferred embodiments of the invention without departing substantially from the spirit and principles of the invention. All such modifications and variations are intended to be included herein within the scope of the present invention, as defined by the following claims.

I claim:

1. A linear blood pump, comprising:
a tube having an inner wall, an inner diameter, an axial length and a constant portion,
said inner wall being designed and arranged such that blood contacting said inner wall may flow though said tube, said inner wall being made of a material selected from the group consisting of pyrolytic graphite and titanium alloys,
said constant portion being designed to extend along at least a part of the axial length of said tube and to have a constant inner diameter; and
at least one check valve, said check valve being arranged in said constant portion, said check valve being designed and arranged to reciprocate in said constant portion.

2. The linear blood pump of claim 1, wherein said titanium alloy is titanium nitride.

3. The linear blood pump of claim 1, further comprising:
a second check valve, said second check valve being arranged in said constant portion, said first check valve being designed and arranged to reciprocate with respect to said second check valve.

4. The linear blood pump of claim 3, wherein said second check valve is designed and arranged to be stationary.

5. The linear blood pump of claim 3, wherein said second check valve is designed and arranged to reciprocate in said constant portion.

6. The linear blood pump of claim 5, wherein said tube is designed to be U-shaped and to include a first straight portion, a second straight portion and a bent portion, said first and second straight portions being interconnected by said bent portion, said first check valve being located in said first straight portion and said second check valve being located in said second straight portion.

7. The linear blood pump of claim 6, wherein said blood pump is designed to be implanted in an organism.

8. The linear blood pump of claim 3, wherein said first and second check valves are designed as heart valves, at least one of said heart valves being connected to a ring, said ring having an outer diameter which is coordinated with the inner diameter of said tube such that said heart valve reciprocates in said tube without tilting.

9. The linear blood pump of claim 1, further comprising at least one sensor, said sensor being designed and arranged to sense the position of said check valve in said constant portion.

10. The linear blood pump of claim 9, further comprising a drive, said drive being designed and arranged to reciprocate said check valve in said constant portion in response to a signal of said sensor.

11. A linear blood pump system, comprising:
a blood pump, said blood pump including
a tube having an inner wall, an inner diameter, an axial length and a constant portion, said inner wall being designed and arranged such that blood contacting said inner wall may flow though said tube, said constant portion being designed to extend along at least a part of the axial length of said tube and to have a constant inner diameter, and
at least one check valve, said check valve being arranged in said constant portion, said check valve being designed and arranged to reciprocate in said constant portion; and
at least one sensor, said sensor being designed and arranged to sense at least one value selected from the group consisting of position, velocity and acceleration of said check valve.

12. The linear blood pump system of claim 11, wherein said sensor is designed as an ultrasonic transceiver unit.

13. The linear blood pump system of claim 11, further comprising a plurality of sensors, said sensors being designed as Hall sensors.

14. The linear blood pump system of claim 11, further comprising a drive, said drive being designed and arranged to reciprocate said check valve in said constant portion in response to a signal of said sensor.

15. The linear blood pump system of claim 14, wherein said drive includes a linear induction motor.

16. The linear blood pump system of claim 15, wherein said linear induction motor is designed as a synchronous motor.

17. The linear blood pump system of claim 11, wherein said inner wall is made of a material selected from the group consisting of pyrolytic graphite and titanium alloys.

18. The linear blood pump system of claim 17, wherein said titanium alloy is titanium nitride.
